(19) United States
(12) Patent Application Publication
(10) Pub. No.: US 2025/0025380 A1
(43) Pub. Date: Jan. 23, 2025

(54) PERSONAL CARE DISSOLVABLE SHEET, COMPOSITIONS, METHODS OF MAKING AND USING THE SAME

(71) Applicant: IPX, LLC, Nashville, TN (US)

(72) Inventor: Kimberly Allison Durrance, Lavinia, TN (US)

(73) Assignee: IPX, LLC, Nashville, TN (US)

(21) Appl. No.: 18/776,567

(22) Filed: Jul. 18, 2024

Related U.S. Application Data
(60) Provisional application No. 63/527,898, filed on Jul. 20, 2023.

(51) Int. Cl. A61K 8/02; A61Q 5/02

(52) U.S. Cl. CPC A61K 8/0216; A61Q 5/02; A61K 2800/5422; A61K 2800/592; A61K 2800/805

(57) ABSTRACT

Personal care dissolvable sheets which can effectively eliminate the use of plastic bottles for packaging and delivery are provided. Solid, dry, lightweight, flexible shampoo sheets, which are activated by water and completely dissolves when in contact with water are also provided. Additionally, compositions and methods for preparing and using shampoo sheets are provided.

### BACKGROUND OF THE INVENTION

### Field of the Invention

The present invention generally relates to a personal care dissolvable sheet, compositions and methods thereto. More particularly, embodiments of the invention relate to a shampoo sheet, and methods of preparing and using the same.

### Description of the Related Art

Traditionally, hair care products, namely, shampoos or conditioners are packed in plastic bottles with about 80-90% of water. Plastics have become essential components of products and packaging, but plastics originate as fossil fuels and emit greenhouse gases from cradle to grave. Plastic waste is doing real harm to ocean ecosystens and animals, amongst other detrimental effects. Plastic pollution reduces our waterways' recreational and aesthetic value, interferes with navigation, and disrupts commercial and recreational fishing. Microplastics also pose a risk to human health.

A personal care sheet offers a convenient technology for delivering compositions, including for skin care, hair care, or cosmetics applications. Such sheets are designed to quickly dissolve upon contact with water or aqueous compositions provided by the end user. An important goal in the industry is to ensure that the performance of these sheets will rival that of their conventional wet counterparts in plastic bottles, while providing the greater portability and convenience of a sheet.

The success of a cosmetic, including personal care sheets, depends in great measure on the way it feels to a user. Thus, speed of dissolution and sensory attributes, such as ease of spreading, speed of absorption, texture, skin moistness, heaviness, amount of grease, amount of tack, quickness of drying, overall hair or skin feel, and overall appearance are critical factors. For example, if the product dissolves too slowly, it becomes gummy and lumpy and does not provide the desired uniform cleansing effect.

In summary, the prior art has not demonstrated a shampoo or other personal care products that can effectively eliminate the use of plastic bottles. Thus, there is an urgent and continuing need for a plastic bottle free personal care sheet or a shampoo sheet that is activated by water and completely dissolves when in contact with water. There is also a need for a solid state, compact, lightweight and ecofriendly personal care sheet. Accordingly, a need exists for a cost-effective personal care product, particularly a shampoo sheet to eliminate the need of plastic bottle for packaging and delivery.

Thus, it is an object of the present invention to meet these needs and address the issues associated the personal care products.

Therefore, it is an object of the present invention to provide a solid, dry, flexible shampoo sheet.

It is another object of the present invention to provide a shampoo sheet, which can eliminate the need for a plastic bottle for packaging and delivery.

It is still another object of the present invention to provide a shampoo sheet that is activated by water and completely dissolves when in contact with water.

It is also an object of the present invention to provide an ecofriendly green alternative to traditional plastic bottled shampoos, reduce carbon impact, and which dissolved to leave no trace of the delivered technology.

It is a further object of the present invention to provide a shampoo sheet that is cost-efficient to produce and distribute.

These needs and other needs are met by the various aspects of the present disclosure.

### SUMMARY OF THE INVENTION

In some embodiments, a personal care dissolvable sheet can include a first anionic surfactant; a second anionic surfactant; a third amphoteric or zwitterionic surfactant; a polyvinyl alcohol or an analog or a derivative; a thickener; an oil; an optional additive; and a suitable carrier, wherein a mixture of polyvinyl alcohol and carrier is heated and filtered to form a filtrate; a mixture of the first anionic surfactant, the second anionic surfactant, the third amphoteric or zwitterionic surfactant, the thickener, the oil and the additive is added to the filtrate and stirred to form a solution; and the solution is poured onto a drying plate or a drying drum to form the personal care dissolvable sheet.

In other embodiments, a method for preparing a personal care dissolvable sheet can include heating a mixture of a polyvinyl alcohol and a carrier until dissolved; filtering the mixture through a mesh to form a filtrate; adding a first anionic surfactant, a second anionic surfactant, a third amphoteric surfactant or zwitterionic surfactant, a thickener, an oil and an additive to the filtrate to form a mixture; stirring the mixture to form a solution; pouring the solution onto a drying plate or a drying drum to form the personal care dissolvable sheet.

In certain embodiments, a method of shampooing hair with a personal care dissolvable sheet can include wetting the hair with water; applying the personal care dissolvable sheet onto the wet hair; foaming the sheet by massaging the hair; and rinsing the hair with water.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate (one) several embodiment(s) of the invention and together with the description, serve to explain the principles of the invention.

FIG. 1 shows square shaped shampoo sheet.

FIG. 2 shows round shaped shampoo sheet.

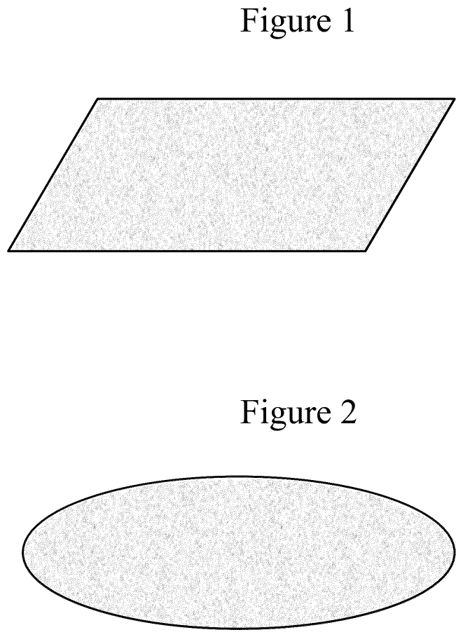

### DETAILED DESCRIPTION OF THE INVENTION

### I. Definitions

Throughout this disclosure, all the scientific and technical terms, unless otherwise indicated, shall have the same meanings as those known to a person skilled in the art. Where there is inconsistency, the definition provided in the present disclosure should be taken.

It should be understood that the detailed description of all materials, processes, examples and drawings are presented for the purposes of illustration, and therefore, unless expressly specified otherwise, are not construed as limitations of the present disclosure.

The following terms are intended to have the meanings presented therewith below and are useful in understanding the description and intended scope of the present invention.

The use of the terms “a”, “an” and “the” and similar referents in the context of describing the subject matter of this application (especially in the context of the following claims) are to be construed to cover both the singular and the plural, unless otherwise indicated herein or clearly contradicted by context.

The term “about” as used herein, refers that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments. Additionally, in phrase “about X to Y,” is the same as “about X to about Y,” that is the term “about” modifies both “X” and “Y.”

The term “surfactant or surface-active agent” as used herein, refers to a chemical that stabilizes mixtures of oil and water by reducing the surface tension at the interface between the oil and water molecules. Surfactants provide cleansing, wetting, foaming and conditioning properties.

The term “additive” as used herein, refers to any substance that is added to achieve or improve a desired effect or properties of the formulation or product. Natural additives are chemical compounds extracted from plants, animals or minerals. Synthetic additives are not extracts, but are the result of a chemical or enzymatic reaction.

The term “carrier” as used herein, refers to a medium, which can be vegetable oils or base oils, water or non-aqueous organic solvents.

The term “thickener” as used herein, refers to a compound is to act a& thickening agent that increase the viscosity of the final product. Most thickening agents are polysaccharides, such as starches or gums, or proteins, such as gelatin.

The term “polymer.” as used herein, includes materials whether made by polymerization of one type of monomer or made by two (i.e., copolymers) or more types of monomers.

The term “fluid” as used herein, includes liquids, gels, emulsions, or suspensions.

The term “mixtures” as used herein, is meant to include a simple combination of materials and any compounds that may result from their combination.

The term “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are used in their inclusive, open-ended, and non-limiting sense.

The term “personal care” as used herein, refers to compositions that are to be topically applied to a person. Examples of personal care compositions include skin care and cosmetic products (e.g., facial cream, moisturizers, lotions, sunscreens, foundation, mascara, eye-liner, lipstick, and the like), nail care products (such as polish and conditioners), and hair care products (including styling gels, hairsprays, shampoos).

The term “personal care dissolvable sheet” as used herein, refers to a readily dissolvable sheet containing a personal care composition. In one embodiment, such a sheet is a shampoo sheet that is activated by water and completely dissolves when in contact with water.

The term “suitable for application to human hair,” as used herein, means that the compositions or components thereof so described are acceptable for use in contact with human hair and the scalp and skin without undue toxicity, incompatibility, instability, allergic response, and the like.

The term “water soluble,” as used herein, means that the material is soluble in water in the present composition. In general, the material should be soluble at 25° C. at a concentration of 0.1% by weight of the water solvent, alternatively at 1%, alternatively at 5%, and alternatively at 15%.

The term “effective,” as that term is used in the specification and/or claims, means adequate to accomplish a desired, expected, or intended result.

Unless otherwise specified, every numerical range in this context intends to include both endpoints and any numbers and sub-ranges falling within said numerical ranges.

Unless specially indicated, all materials and agents used in the present disclosure are commercially available.

### II. Personal Care Dissolvable Sheet

According to various aspects of the disclosure, the invention relates to a personal care dissolvable sheet, compositions, and methods for making and using the same. In further aspects, one embodiment of the present invention relates to a shampoo sheet, compositions of the sheet according to the present invention and methods for making and using the same.

It was unexpectedly and surprisingly discovered that a personal care dissolvable sheet, provided in one embodiment of the invention can effectively eliminate the use of plastic bottles for packaging and delivery, while allowing the user to apply and use to clean the hair as effectively as the conventional shampoo delivered in a plastic bottle.

It was also unexpectedly and surprisingly discovered that a personal care sheet particularly a shampoo sheet, can be provided in the form of solid, dry, flexible sheet, which is activated by water and completely dissolves when in contact with water.

It was further unexpectedly and surprisingly discovered that the present invention provides an ecofriendly alternative to traditional plastic-bottled packed shampoos provided in a plastic bottle, reduces carbon impact and leaves no trace of technology.

It was also unexpectedly and surprisingly discovered that a personal care dissolvable sheet in accordance with the present invention can be produced as a compact, light-weight sheet that cleanses the hair remarkably well and is cost-efficient to produce and distribute.

In some embodiments, a personal care dissolvable sheet can include a first anionic surfactant; a second anionic surfactant; a third amphoteric or zwitterionic surfactant; a polyvinyl alcohol or an analog or a derivative; a thickener; an oil; an optional additive; and a suitable carrier, wherein a mixture of polyvinyl alcohol and carrier is heated and filtered to form a filtrate; a mixture of the first anionic surfactant, the second anionic surfactant, the third amphoteric or zwitterionic surfactant, the thickener, the oil and the additive is added to the filtrate and stirred to form a solution; and the solution is poured onto a drying plate or a drying drum to form the personal care dissolvable sheet.

The shampoo composition of the present invention can include one or more surfactants. The surfactant component is included in shampoo composition of the present invention to provide cleansing performance. In one embodiment, the first surfactant can include an anionic surfactant. In another embodiment, the second surfactant can include an anionic surfactant. In other embodiment, the third surfactant can include an amphoteric or a zwitterionic surfactant.

Suitable anionic surfactants include those which are known for use in hair care or other personal care shampoo compositions. In at least one embodiment, the anionic surfactant is selected from the group consisting of (C₁₀-C₂₀)-alkyl and alkylene carboxylates, alkyl ether carboxylates, fatty alcohol sulfates, fatty alcohol ether sulfates, alkylamide sulfates and sulfonates, fatty acid alkylamide polyglycol ether sulfates, alkanesulfonates and hydroxyalkanesulfonates, olefinsulfonates, acyl esters of isethionates, α-sulfo fatty acid esters, alkylbenzenesulfonates, alkylphenol glycol ether sulfonates, sulfosuccinates, sulfosuccinic monoesters and diesters, fatty alcohol ether phosphates, protein/fatty acid condensation products, alkyl monoglyceride sulfates and sulfonates, alkylglyceride ether sulfonates, fatty acid methyltaurides, fatty acid sarcosinates, sulforicinoleates, acylglutamates, and mixtures thereof. The anionic surfactants (and their mixtures) can be used in the form of their water-soluble or water-dispersible salts, examples being the sodium, potassium, magnesium, ammonium, mono-, di-, and triethanolammonium, and analogous alkylammonium salts. In at least one embodiment, the anionic surfactant is the salt of an anionic surfactant comprising 12 to 14 carbon atoms. In at least one embodiment, the anionic surfactant is selected from the group consisting of sodium lauryl sulfate, sodium laureth sulfate, sodium tridecyl sulfate, sodium trideceth sulfate, sodium myristyl sulfate, sodium myreth sulfate, and mixtures thereof. Typical anionic surfactants for use in compositions of the invention include sodium oleyl succinate, ammonium lauryl sulphosuccinate, sodium lauryl sulphate, sodium lauryl ether sulphate, sodium lauryl ether sulphosuccinate, ammonium lauryl sulphate, ammonium lauryl ether sulphate, sodium dodecyl benzene sulphonate, triethanolamine dodecylbenzene sulphonate, sodium cocoyl isethionate, sodium lauryl isethionate, lauryl ether carboxylic acid and sodium N-lauryl sarcosinate. Preferred anionic surfactants is a combination of sodium lauryl sulphate and sodium laureth sulfate.

In some embodiments, the total amount of anionic surfactant ranges from about 1 wt. % to about 50 wt. %, preferably from about 5 wt. % to about 40 wt. % and more preferably from about 10 wt. % to about 30 wt. %.

In certain embodiments, the anionic surfactant is selected from the group consisting of ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, monoethanolamine cocoyl sulfate, sodium trideceth sulfate, sodium tridecyl sulfate, sodium methyl lauroyl taurate, sodium methyl cocoyl taurate, sodium lauroyl isethionate, sodium cocoyl isethionate, sodium laurethsulfosuccinate, sodium laurylsulfosuccinate, sodium tridecyl benzene sulfonate, sodium dodecyl benzene sulfonate, and mixtures thereof.

In one embodiment, the first anionic surfactant is sodium laureth sulfate.

In another embodiment, the second anionic surfactant is sodium lauryl sulfate.

In other embodiment, the third surfactant can include an amphoteric surfactant or the zwitterionic surfactant.

In certain embodiments, the amphoteric surfactant or the zwitterionic surfactant is selected from the group consisting of sodium 3-dodecyl-aminopropionate, sodium 3-dodecylaminopropane sulfonate, sodium lauryl sarcosinatc. N-alkyltaurines, coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, coco dimethyl sulfopropyl betaine, stearyl dimethyl sulfopropyl betaine, lauryl dimethyl sulfoethyl betaine, lauryl bis-(2-hydroxyethyl) sulfopropyl betaine, cocodimethylcarboxymethylbetaine, lauryldimethylcarboxymethylbetaine, lauryldimethylalphacarboxyethylbetaine, cetyldimethylcarboxymethylbetaine, oleyldimethylgammacarboxypropylbetaine, laurylbis(2-hydroxypropyl)alphacarboxyethylbetaine and mixtures thereof.

In at least one embodiment, the amphoteric surfactants or the zwitterionic surfactants are selected from the group consisting of N—(C₁₂-C₁₈)-alkyl-β-aminopropionates and N—(C₁₂-C₁₈)-alkyl-β-iminodipropionates as alkali metal salts and mono-di-, and trialkylammonium salts; N-acylaminoalkyl-N,N-dimethylacetobetaine, preferably N—(C₈-C₁₈)-acylaminopropyl-N,N-dimethylacetobetaine, (C₁₂-C₁₈)-alkyl-dimethyl-sulfopropylbetaine, amphosurfactants based on imidazoline (trade name: Miranol®. Steinapoe), preferably the sodium salt of 1-β-carboxymethyloxyethyl)-1-(carboxymethyl)-2-laurylimidazolinium; amine oxide, e.g., (C₁₂-C₁₈)-alkyl-dimethyl-amine oxide, fatty acid amidoalkyldimethylamine oxide, and mixtures thereof.

In other embodiments, the third amphoteric surfactant or the zwitterionic surfactant is a betaine surfactant.

In another embodiment, the betaine surfactant is a cocamidopropyl betaine.

Optionally, the betaine surfactant or the zwitterionic surfactant is selected from C₈- to C₁₈-alkylbetaines. In at least one embodiment, the betaine surfactant is selected from the group consisting of cocodimethylcarboxymethylbetaine, lauryldimethylcarboxymethylbetaine, lauryldimethylalphacarboxyethylbetaine, cetyldimethylcarboxymethylbetaine, oleyldimethylgammacarboxypropylbetaine and laurylbis(2-hydroxypropyl) alphacarboxyethylbetaine and combinations thereof. Optionally, the betaine surfactant is selected from C₈- to C₁₈-sulfobetaines. In at least one embodiment, the betaine surfactant is selected from the group consisting of cocodimethylsulfopropylbetaine, stearyldimethylsulfopropylbetaine, lauryldimethyl-sulfoethylbetaine, laurylbis(2-hydroxyethyltsulfopiopylbetaine, and combinations thereof. Optionally, the betaine surfactant is selected from carboxyl derivatives of imidazole, the C8- to C18-alkyldimethylammonium acetates, the C₈- to C₁₈-alkyldimethylcarbonylmethylanimonium salts, and the C₈- to C₁₈-fatty acid alkylamidobetaines, and mixtures thereof. Optionally, the C₈- to C₁₈-fatty acid alkylamidobetaine is selected from coconut fatty acid amidopropylbetaine, N-coconut fatty acid amnidoethyl-N-[2-carboxymethoxy)ethyl]glycerol (CTFA name: Cocoamphocarboxyglycinate), and mixtures thereof. A particularly preferred amphoteric or zwitterionic surfactant is cocamidopropyl betaine.

In some embodiments, the total amount of the amphoteric surfactant or the zwitterionic surfactant ranges from about 0.5 wt. % to about 20 wt. %, preferably from about 1 wt. % to about 10 wt. % and more preferably from about 3 wt. % to about 6 wt. %.

In some embodiments, the sheet can further include a nonionic surfactant.

In other embodiments, the nonionic surfactant is selected from the group consisting of lauryl dimethylamine oxide, cocodimethylamine oxide, cocoamidopropylamine oxide, laurylamidopropyl amine oxide, cocomonoethanol amide, cocodiethanol amide, lauroylmonoethanol amide, alkanoyl isopropanol amides, cetyl alcohol, oleyl alcohol, 2-hydroxyalkyl methyl ethers, glucose amides, alkyl polyglucosides, sucrose cocoate, sucrose laurate, alkanolamides, ethoxylated alcohols, glyceryl monohydroxystearate, isosteareth-2, trideceth-3, hydroxystearic acid, propylene glycol stearate, PEG-2 stearate, sorbitan monostearate, glyceryl laurate, laureth-2, cocamide monoethanolamine, lauramide monoethanolamine, and mixtures thereof.

In some embodiments, the sheet can further include a cationic surfactant.

In other embodiments, the cationic surfactant is selected from the group consisting of bis-hydroxyethyl lauryl amine, lauryl dimethylamine, lauroyl dimethyl amidopropyl amine, cocoylamidopropyl amine, lauryl trimethylammonium chloride, lauroylamidopropyl trimethyl ammonium chloride, and mixtures thereof.

Examples are thickeners include hydroxyethyl-cellulose, hydroxypropylcellulose, and carboxymethylcellulose, guar gum, such as hydroxypropylguar, gums of microbial origin, such as xanthan gum and scleroglucan gum, and synthetic thickeners, such as crosslinked homo- or copolymers of acrylic acid and/or of acrylamidopropanesulphonic acid. Other thickeners include Zea mays corn starch, kuzu powder (Japanese arrowroot), xanthum gum, gelatin, polyvinyl alcohol or an analog or a derivative or combinations thereof.

While oils and oil derivatives, synthetic as well as natural, can be employed as other ingredients as is commonly done in skin and hair care products to impart conditioning properties to hair, tea tree oil, in the present invention, functions in excess of its conditioning properties and appears to produce a more that additive result when used in combination with antidandruff agents. Commonly used oils include canola, olive, castor, avocado, almond, soybean, grape seed, hazelnut, mineral and others. A particularly preferred oil is a mineral oil.

The total amount of the oil ranges from about 0.01 wt. % to about 5 wt. %, preferably from about 0.03 wt. % to about 4 wt. % and more preferably from about 0.05 wt. % to about 2.5 wt. %.

In one embodiment, the oil is a mineral oil.

In certain embodiments, the additive is selected from the group consisting of conditioning agent, antidandruff agent, dyes, pigments, perfumes, foam booster, preservative, pearlescent agent, skin active agent, sunscreen, UV absorber, vitamin, protein, emulsifier, antibacterial agent, antifungal agent and mixtures thereof.

Conditioning agents are used in shampoos, especially in the conditioning shampoos, to provide conditioning effects to the hair. A large number of materials would be suitable conditioning agents for shampoos. Typically, a blend of conditioning agents is employed, as is typical in the shampoo formulating art.

The use of silicon compounds as conditioning agents in shampoos is well known, and they are employed in their intended use. The volatile silicone normally is a low molecular weight polydimethylsiloxane, however a low molecular weight polysiloxane including phenyl substituents also is useful in the present invention. Furthermore, the low molecular weight polydimethysiloxane compound can be a linear or a cyclic polydimethysiloxane compound. The volatile polydimethylsiloxane compound provides lubrication and imparts hair-conditioning properties to wet hair, and has sufficient volatility to slowly volatilize from the hair such that a residual buildup of silicone compound is not present on dry hair. The silicone compounds that are commonly used in shampoos are dimethicone, dimethiconepolyol, and silicone gum. Dimethicone belongs to the class of compounds known as polydimethylsilooxanes. Dimethicone coplyol is a derivative of dimethicone in which polyether side chains replace some of the methyl groups. Silicone gum is mixed high molecular weight (from 200,000 to 1,000,000) dimethicone compound that is gum like in nature.

Another suitable water-insoluble conditioning compound that can be included in the composition of the present invention is a nonvolatile hydrocarbon, such as mineral oil. The nonvolatile hydrocarbons provide many of the same benefits as the silicone conditioning agents, and can be included in the composition with a silicone conditioning agent. Other oils such as almond oil, avocado oil, sheabutter, aloe vera, etc., can also be used.

The use of cationic polymers, which have quaternized nitrogen attached to the backbone of the polymer, in hair care products are well known. Being cationic, these polymers are also substantive to hair and are able to impart conditioning attributes to the hair. Suitable cationic polymers include, for example, copolymers of vinyl monomers having cationic amine or quaternary ammonium functionalities with water soluble spacer monomers such as acrylamide, methacrylamide, alkyl and dialkylmethacrylamide, alkyl acrylate, alkyl methacrylate, vinyl caprolactone, and vinyl pyrrolidone. The alkyl and dialkyl substituted monomers preferably have C1-C7 alkyl groups, more preferably C1-C3 alkyl groups. Other suitable spacer monomers include vinyl esters, vinyl alcohol (made by the hydrolysis of vinyl acetate,) ethylene glycol, propylene glycol, and maleic anhydride.

The antidandruff agent is normally a water-insoluble hair treatment compound and usually is a particulate compound that is capable of relieving the symptoms of dandruff and is substantive to hair and scalp to impart residual antidandruff properties between shampoos. Examples of particulate compounds exhibiting antidandruff properties include, but not limited to, elemental sulfur, salicylic acid, seleneium sulfide, coal tar preparations, zinc pyrithione, a water-insolublel-hydroxy pyridone, an azole antimycotic, and undecylenic acid and mixtures thereof. Particularly advantageous antidandruff agents useful in the shampoo composition of the present invention are zinc pyrithione and elemental sulfur. Zinc pyrithione is the zinc complex of 2-pyridinethiol-1-oxide, and is available commercially from Arch Chemicals, under the brand name of Omadine®. Useful sulfur containing compounds include elemental sulfur and selenium sulfide of sufficient purity and particle size to function as an antidandruff agent, as is well known to those skilled in the art.

The total amount of the antidandruff agent ranges from about 0.01 wt. % to about 5 wt. %, preferably from about 0.03 wt. % to about 2 wt. % and more preferably from about 0.05 wt. % to about 1 wt. %.

Suitable cationic dyes are Basic Blue 6, Basic Blue 7, Basic Blue 9, Basic Blue 26, Basic Blue 41, Basic Blue 99, Basic Brown 4, Basic Brown 16, Basic Brown 17, Natural Brown 7, Basic Green 1, Basic Red 2, Basic Red 12 Basic Red 22, Basic Red 76, Basic Violet 1, Basic Violet 2, Basic Violet 3, Basic Violet 10, Basic Violet 14 and Basic Yellow 57.

The total amount of the cationic dye ranges from about 0.0001 wt. % to about 2 wt. %, preferably from about 0.0001 wt. % to about 1.5 wt. % and more preferably from about 0.0001 wt. % to about 1 wt. %.

Anionic dyes may as well be used in combination with cationic direct dyes at minor quantities. Suitable anionic dyes are Acid Black 1, Acid Blue 1, Acid Blue 3, Food Blue 5, Acid Blue 7, Acid Blue 9, Acid Blue 74, Acid Orange 3, Acid Orange 6, Acid Orange 7, Acid Orange 10, Acid Red 1, Acid Red 14, Acid Red 18, Acid Red 27, Acid Red 50, Acid Red 52, Acid Red 73, Acid Red 87, Acid Red 88, Acid Red 92, Acid Red 155, Acid Red 180, Acid Violet 9, Acid Violet 43, Acid Violet 49, Acid Yellow 1, Acid Yellow 23, Acid Yellow 3, Food Yellow No. 8, D&C Brown No. 1, D&C Green No. 5, D&C Green No. 8, D&C Orange No. 4, D&C Orange No. 10, D&C Orange No. 11, D&C Red No. 21, D&C Red No. 27, D&C Red No. 33, D&C Violet 2, D&C Yellow No. 7, D&C Yellow No. 8, D&C Yellow No. 10, FD&C Red 2, FD&C Red 40, FD&C Red No. 4, FD&C Yellow No. 6, FD&C Blue 1, Food Black 1, Food Black 2, Disperse Black 9 and Disperse Violet 1 and their alkali metal salts such as sodium, potassium.

The total amount of the anionic dyes ranges from about 0.0001 wt. % to about 2 wt. %, preferably from about 0.0001 wt. % to about 1.5 wt. % and more preferably from about 0.0001 wt. % to about 1 wt. %.

A shampoo sheet can optionally include pigments such as inorganic, organic, organometallic, nitroso, monoazo, disazo, carotenoid, triphenyl methane, triaryl methane, xanthene, quinoline, oxazine, azine, anthraquinone, indigoid, thioindigoid, quinacridone, phthalocyanine, botanical, natural colors, including: water soluble components such as those having C. I. Names.

Preservatives are required in shampoos to safeguard against microbial action that could cause spoilage of the product. Commonly used preservatives in shampoos include methyl and propyl para-hydroxybenzoates alone or in combination with imidazolinyl urea, methylisothioazolinone, methyloldiethylhydantoin (DMDMH), methchloroisothiazolinone, and N-(3-chloroallyl)hexaminium chloride (Quaternium 15).

Additional examples of suitable preservatives include benzyl alcohol, piroctone olamine, phenoxyethanol, parabens, pentanediol, benzoic acid/sodium benzoate, sorbic acid/potassium sorbate, and other organic acids used to provide antimicrobial protection. Preservation boosting ingredients include anisic acid, lactic acid, sorbitan caprylate, ethylhexylglycerin, caprylyl glycol, octanediol, and similar substances. The total amount of the preservative ranges from about 0.01 wt. % to about 5 wt. %, preferably from about 0.03 wt. % to about 4 wt. % and more preferably from about 0.05 wt. % to about 1 wt. % b.

Pearlescent agent, another desirable component of the shampoos herein is a pearlescent material. Such materials are well known ion the art and include bismuth oxychloride, stearic monoethanolamide, ethylene glycol mono stearate or distreate, guanine and titanium dioxide coated mica. A pearlescent material is generally present at a level of from about 0.1 wt. % to about 6 wt. %, preferably from about 0.5 wt. % to about 3 wt. %.

In some embodiments, the antifungal agent is selected from the group consisting of ketoconazole, oxiconazole, bifonazole, butoconazole, cloconazole, clotrimazole, econazole, enilconazole, fenticonazole, isoconazole, miconazole, sulconazole, tioconazole, fluconazole, itraconazole, terconazole, naftifine and terbinafine, zinc pyrithione, octopirox, and combinations thereof.

In other embodiments, the total amount of the antifungal agent ranges from about 0.1 wt. % to about 1 wt. %.

The carrier of the shampoo composition is predominantly water, but non-aqueous solvents can also be used to solubilize composition ingredients that are not sufficiently soluble in water, to adjust the viscosity of the composition or to act as humectant. Suitable solvents include polyols, like glycols, glycerol or mixtures thereof. The optional non-aqueous solvents should not adversely affect the ability of the composition to cleanse and treat the hair and scalp or adversely affect aesthetic appeal of the formulation. The total amount of the non-aqueous solvent ranges from about 0 wt. % to about 5 wt. %.

In one embodiment, the suitable carrier is water.

In certain embodiments, the amount of water ranges from about 1 wt. % to about 5 wt. % of the sheet.

In another embodiment, the personal care dissolvable sheet is a shampoo sheet. The shampoo sheet can be of any size or shape. The shampoo sheet can be of colorless or of any color.

### II. Method for Preparing a Personal Care Dissolvable Sheet

In other embodiments, a method for preparing a personal care dissolvable sheet can include heating a mixture of a polyvinyl alcohol and a carrier until dissolved; filtering the mixture through a mesh to form a filtrate; adding a first anionic surfactant, a second anionic surfactant, a third amphoteric surfactant or zwitterionic surfactant, a thickener, an oil and an additive to the filtrate to form a mixture; stirring the mixture to form a solution; pouring the solution onto a drying plate or a drying drum to form the personal care dissolvable sheet.

In one embodiment, the suitable carrier is water.

In certain embodiments, the amount of water ranges from about 1 wt. % to about 5 wt. % of the sheet.

### IV. Method of Treating Hair with a Personal Care Dissolvable Sheet

In certain embodiments, a method of shampooing hair with a personal care dissolvable sheet can include wetting the hair with water; applying the personal care dissolvable sheet onto the wet hair; foaming the sheet by massaging the hair; and rinsing the hair with water. These steps can be repeated as many times as desired to achieve the desired cleansing and conditioning benefit.

### EXAMPLES

In order to provide a better understanding of the foregoing discussion, the following non-limiting examples are provided. Although the examples may be directed to specific embodiments, they are not to be viewed as limiting the invention in any specific respect. All parts, proportions, and percentages are by weight unless otherwise indicated.

| Example 1 | (grams) |
|---|---|
| Zea mays (corn) starch | 39.00 |
| Sodium laureth sulfate | 23.00 |
| Polyvinyl alcohol | 13.00 |
| Sodium lauryl sulfate | 6.00 |
| Diethanolamine | 6.00 |
| Cocamidopropyl betaine | 6.00 |
| Water | 4.50 |
| Mineral oil | 2.0 |
| Additive | 0.5 |

A mixture of polyvinyl alcohol and water was heated until it is completely dissolved and filtered to collect the filtrate. A mixture of sodium laureth sulfate, sodium lauryl sulfate, cocamidopropyl betaine, Zea mays (corn) starch, diethanolamine, mineral oil and additive(s) was added to the filtrate and stirred to form a solution. The solution was poured onto a drying plate or a drying drum to form the personal care dissolvable sheet. The product is solid, compact, lightweight, flexible and can be cut into any size or shape.

| Example 2 | (grams) |
|---|---|
| Kuzu powder (Japanese arrowroot) | 39.00 |
| Sodium laureth sulfate | 23.00 |
| Polyvinyl alcohol | 13.00 |
| Sodium lauryl sulfate | 6.00 |
| Diethanolamine | 6.00 |
| Cocamidopropyl betaine | 6.00 |
| Water | 4.50 |
| Mineral oil | 2.0 |
| Additive | 0.5 |

A mixture of polyvinyl alcohol and water was heated until it is completely dissolved and filtered to collect the filtrate. A mixture of sodium laureth sulfate, sodium lauryl sulfate, cocamidopropyl betaine, kuzu powder (Japanese arrowroot), diethanolamine, mineral oil and additive(s) was added to the filtrate and stirred to form a solution. The solution was poured onto a drying plate or a drying drum to form the personal care dissolvable sheet. The product is solid, compact, lightweight, flexible and can be cut into any size or shape.

| Example 3 | grams |
|---|---|
| Xanthum gum | 39.00 |
| Sodium laureth sulfate | 23.00 |
| Polyvinyl alcohol | 13.00 |
| Sodium lauryl sulfate | 6.00 |
| Diethanolamine | 6.00 |
| Cocamidopropyl betaine | 6.00 |
| Water | 4.50 |
| Mineral oil | 2.0 |
| Additive | 0.5 |

A mixture of polyvinyl alcohol and water was heated until it is completely dissolved and filtered to collect the filtrate. A mixture of sodium laureth sulfate, sodium lauryl sulfate, cocamidopropyl betaine, xanthum gum, diethanolamine, mineral oil and additive(s) was added to the filtrate and stirred to form a solution. The solution was poured onto a drying plate or a drying drum to form the personal care dissolvable sheet. The product is solid, compact, lightweight, flexible and can be cut into any size or shape.

| Example 4 | (grams) |
|---|---|
| Gelatin | 29.00 |
| Xanthum gum | 10.00 |
| Sodium laureth sulfate | 23.00 |
| Polyvinyl alcohol | 13.00 |
| Sodium lauryl sulfate | 6.00 |
| Diethanolamine | 6.00 |
| Cocamidopropyl betaine | 6.00 |
| Water | 4.50 |
| Mineral oil | 2.0 |
| Additive | 0.5 |

A mixture of polyvinyl alcohol and water was heated until it is completely dissolved and filtered to collect the filtrate. A mixture of sodium laureth sulfate, sodium lauryl sulfate, cocamidopropyl betaine, gelatin, xanthum gum, diethanolamine, mineral oil and additive(s) was added to the filtrate and stirred to form a solution. The solution was poured onto a drying plate or a drying drum to form the personal care dissolvable sheet. The product is solid, compact, lightweight, flexible and can be cut into any size or shape.

| Example 5 | (grams) |
|---|---|
| Zea mays (corn) starch | 39.00 |
| Sodium laureth sulfate | 23.00 |
| Aqua Sys GP | 13.00 |
| Sodium lauryl sulfate | 6.00 |
| Diethanolamine | 6.00 |
| Cocamidopropyl betaine | 6.00 |
| Water | 4.50 |
| Mineral oil | 2.0 |
| Additive | 0.5 |

A mixture of Aqua Sys GP and water was heated until it is completely dissolved and filtered to collect the filtrate. A mixture of sodium laureth sulfate, sodium lauryl sulfate, cocamidopropyl betaine, Zea mays (corn) starch, diethanolamine, mineral oil and additive(s) was added to the filtrate and stirred to form a solution. The solution was poured onto a drying plate or a drying drum to form the personal care dissolvable sheet. The product is solid, compact, lightweight, flexible and can be cut into any size or shape.

While in the foregoing specification this invention has been described in relation to certain embodiments thereof, and many details have been put forth for the purpose of illustration, it will be apparent to those skilled in the art that the invention includes additional embodiments and that certain of the details described herein can be varied considerably without departing from the basic principles of the invention. Although we have described the preferred embodiments for implementing our invention, it will be understood by those skilled in the art to which this disclosure is directed that modifications and additions may be made to our invention without departing from its scope.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

What is claimed is:

1. A personal care dissolvable sheet, comprising:
a first anionic surfactant;
a second anionic surfactant;
a third amphoteric or zwitterionic surfactant;
a polyvinyl alcohol or an analog or a derivative;
a thickener;
an oil;
an optional additive; and
a suitable carrier,
wherein a mixture of polyvinyl alcohol and carrier is heated and filtered to form a filtrate;
a mixture of the first anionic surfactant, the second anionic surfactant, the third amphoteric or zwitterionic surfactant, the thickener, the oil and the additive is added to the filtrate and stirred to form a solution; and
the solution is poured onto a drying plate or a drying drum to form the personal care dissolvable sheet.

2. The sheet of claim 1, wherein the anionic surfactant is selected from the group consisting of ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, monoethanolamine cocoyl sulfate, sodium trideceth sulfate, sodium tridecyl sulfate, sodium methyl lauroyl taurate, sodium methyl cocoyl taurate, sodium lauroyl isethionate, sodium cocoyl isethionate, sodium laurethsulfosuccinate, sodium laurylsulfosuccinate, sodium tridecyl benzene sulfonate, sodium dodecyl benzene sulfonate, and mixtures thereof.

3. The sheet of claim 2, wherein the first anionic surfactant is sodium laureth sulfate.

4. The sheet of claim 2, wherein the second anionic surfactant is sodium lauryl sulfate.

5. The sheet of claim 1, wherein the amphoteric surfactant or the zwitterionic surfactant is selected from the group consisting of sodium 3-dodecyl-aminopropionate, sodium 3-dodecylaminopropane sulfonate, sodium lauryl sarcosinatc, N-alkyltaurines, coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, coco dimethyl sulfopropyl betaine, stearyl dimethyl sulfopropyl betaine, lauryl dimethyl sulfoethyl betaine, lauryl bis-(2-hydroxyethyl) sulfopropyl betaine, cocodimethylcarboxymethylbetaine, lauryldimethylcarboxymethylbetaine, lauryldimethylalphacarboxyethylbetaine, cetyldimethylcarboxymethylbetaine, oleyldimethylgammacarboxypropylbetaine, laurylbis(2-hydroxypropyl) alphacarboxyethylbetaine and mixtures thereof.

6. The sheet of claim 5, wherein the third amphoteric surfactant or the zwitterionic surfactant is a betaine surfactant.

7. The sheet of claim 6, wherein the betaine surfactant is a cocamidopropyl betaine.

8. The sheet of claim 1, further comprising a nonionic surfactant.

9. The sheet of claim 8, wherein the nonionic surfactant is selected from the group consisting of lauryl dimethylamine oxide, cocodimethylamine oxide, cocoamidopropylamine oxide, laurylamidopropyl amine oxide, cocomonoethanol amide, cocodiethanol amide, lauroylmonoethanol amide, alkanoyl isopropanol amides, cetyl alcohol, oleyl alcohol, 2-hydroxyalkyl methyl ethers, glucose amides, alkyl polyglucosides, sucrose cocoate, sucrose laurate, alkanolamides, ethoxylated alcohols, glyceryl monohydroxystearate, isosteareth-2, trideceth-3, hydroxystearic acid, propylene glycol stearate, PEG-2 stearate, sorbitan monostearate, glyceryl laurate, laureth-2, cocamide monoethanolamine, lauramide monoethanolamine, and mixtures thereof.

10. The sheet of claim 1, further comprising a cationic surfactant.

11. The sheet of claim 10, wherein the cationic surfactant is selected from the group consisting of bis-hydroxyethyl lauryl amine, lauryl dimethylamine, lauroyl dimethyl amidopropyl amine, cocoylamidopropyl amine, lauryl trimethylammonium chloride, lauroylamidopropyl trimethyl ammonium chloride, and mixtures thereof.

12. The sheet of claim 1, wherein the oil is a mineral oil.

13. The sheet of claim 1, wherein the additive is selected from the group consisting of conditioning agent, antidandruff agent, dyes, pigments, perfumes, foam booster, preservative, pearlescent agent, skin active agent, sunscreen, UV absorber, vitamin, protein, emulsifier, antibacterial agent, antifungal agent and mixtures thereof.

14. The sheet of claim 1, wherein the suitable carrier is water.

15. The sheet of claim 14, wherein the amount of water ranges from about 1 wt. % to about wt. 5% of the sheet.

16. The sheet of claim 1, wherein the sheet is a shampoo sheet.

17. A method for preparing a personal care dissolvable sheet, comprising:
heating a mixture of a polyvinyl alcohol and a carrier until dissolved;
filtering the mixture through a mesh to form a filtrate;
adding a first anionic surfactant, a second anionic surfactant, a third amphoteric surfactant or zwitterionic surfactant, a thickener, an oil and an additive to the filtrate to form a mixture;
stirring the mixture to form a solution;
pouring the solution onto a drying plate or a drying drum to form the personal care dissolvable sheet.

18. The method of claim 17, wherein the carrier is water.

19. The method of claim 18, wherein the amount of water ranges from about 1 wt. % to about 5 wt. % of the sheet.

20. A method of shampooing hair with a personal care dissolvable sheet, comprising:
wetting the hair with water;
applying the personal care dissolvable sheet onto the wet hair;
foaming the sheet by massaging the hair; and
rinsing the hair with water.
